(19) United States
(12) Patent Application Publication
(10) Pub. No.: US 2005/0215659 A1
(43) Pub. Date: Sep. 29, 2005

(54) Dispersions of nanoscale, non-agglomerated particles for use in dental materials

(75) Inventors: Klaus Ruppert, Maintal (DE); Andreas Grundler, Wuppertal (DE); Albert Erdrich, Bad Nauheim (DE)

Correspondence Address: Kurt G. Briscoe;Norris, McLaughlin & Marcus P.A., 18th Floor, 875 Third Avenue, New York, NY, 10022, US

(73) Assignee: Heraeus Kulzer GmbH, Hanau (DE)

(21) Appl. No.: 11/061,787

(22) Filed: Feb. 18, 2005

(30) Foreign Application Priority Data
Feb. 18, 2004 (DE) 10 2004 008 206.5

(51) Int. Cl. A61K 6/08

(52) U.S. Cl. 523/116

(57) ABSTRACT

Dental materials containing an aqueous and/or organic solvent or dispersion with nanoparticles dispersed in it that have an average particle size of 1 to <10 nm and are not agglomerated are particularly suitable for use as filling composites, fastening cement, prostheses base materials, crown veneer composites and bridge veneer composites or adhesives. Preferably the dental materials in accordance with the invention contain SiO₂ and/or ZrO₂ and/or Al₂O₃ and/or TiO₂ nanoparticles.

The invention concerns dental materials comprising dispersions of nanoscale, non-agglomerated particles.

It has already been suggested to use nanoscale, non-agglomerated silicic acid particles with particle sizes of more than 10 nm in organic dispersions in the manufacturing of dental materials. Thus, the traditionally used strongly agglomerated pyrogenic silicic acids have been at least partly replaced thus improving properties such as mechanical strength and transparency. (EP 803 240 A2, DE 196 17 931 A1, U.S. Pat. No. 5,036,006).

It is now found that nanoparticles with an average particle size of 1 to <10 nm bring about surprisingly favorable properties in dental materials.

The nanoparticles are preferably inorganic materials. The invention particularly deals with inorganic materials that are hardly soluble or insoluble in water, particularly oxides as they are used in dental ceramics, e.g., SiO₂, ZrO₂, TiO₂, Al₂O₃, ZnO, CeO₂, La₂O₃, Y₂O₃ or mixtures or mixed oxides thereof.

Nanoparticles can be used preferably alone or also in combination with a small portion of conventional agglomerated fillers (such as, e.g., precipitated silicic acid or pyrogenic silicic acid) or dental glasses.

The invention thus concerns dental materials comprising an aqueous and/or organic solvent or dispersion with nanoparticles that are dispersed in it, that have an average particle size of 1 to <10 nm, particularly 1 to 8 nm and most particularly 2 to 6 nm and are not agglomerated. It is particularly preferred that these particles comprise SiO₂ and/or ZrO₂, and/or Al₂O₃, and/or TiO₂ or mixed oxides thereof. The particles are preferably incorporated into the dental materials as dispersions. The invention particularly deals with monodisperse particles.

In one design form of the invention these nanoparticles are not nanoparticles comprising ZrO₂-particles, TiO₂-particles or Al₂O₃-particles with an average particle size of 1 to <10 nm.

In organic dispersions, the nanoparticles usually are present in hydrophobized form. For this purpose, for example, known silanizing agents such as silane A-174 (Gamma-methacryloxypropyltrimethoxysilane, Union Carbide Corp.) are used. However, even other types of surface modification are possible. It is preferred to use dispersions of nanoparticles in a monomer or a monomer mixture for manufacturing dental materials in accordance with the invention. The further components are then mixed into this mixture.

The dental materials in accordance with the invention have the following advantages:
very high transparency;
clear improvement of the mechanical properties as opposed to materials containing no or larger nanoparticles;
lower viscosity due to which larger filler contents are possible.

Monomers or mixtures thereof are used advantageously as dispersions. The monomers considered for use are those that are conventionally used in the dental field. Examples are monofunctional monomers that are capable of radical polymerization such as mono(meth)acrylates, methyl-, ethyl-, butyl-, benzyl-, furfuryl- or phenyl(meth)acrylate. Polyfunctional monomers include polyfunctional acrylates and/or methacrylates such as, for example, bisphenol-A-di(meth)acrylate, Bis-GMA (an addition product made of methacryl acid and bisphenol-A-diglycidylether), UDMA (an addition product made of 2-hydroxyethylmethacrylate and 2,2,4-hexamethylene diisocyanate), di-, tri- or tetraethylene glycol di(meth)acrylate, decanediol di(meth)acrylate, dodecanediol di(meth)acrylate, hexyldecanediol di(meth)acrylate, trimethylolpropane tri(meth)acrylate, pentaerythrittetra(meth)acrylate and butanediol di(meth)acrylate.

Examples of fillers considered for usage besides the oxides mentioned above (TiO₂, ZrO₂, Al₂O₃, SiO₂) are metal oxides such as tin oxide, metal sulfates, other oxides of the subgroups of the Periodic Table, fluoride-releasing substances, dental glasses, pyrogenic or precipitated silicic acids, dental glasses such as aluminosilicate glasses, fluoroaluminosilicate glasses, strontium silicate, strontium borosilicate, lithium silicate, lithium aluminum silicate, amorphous silicic acids, phyllosilicates, zeolites, amorphous spherical fillers based on oxides or mixed oxides (SiO₂, ZrO₂ and/or TiO₂), metal oxides with primary particle sizes of approximately 40 to 300 nm, splitter polymers with particle sizes of 10-100 μm (cp. R. Janda, Kunststoffverbundsysteme, VCH Verlagsgesellschaft, Weinheim, 1990, Page 225 ff.) or mixtures thereof. In addition, strengthening agents such as glass fibers, polyamide fibers or carbon fibers can be incorporated.

The filler content strongly depends on the intended use. Cements contain preferably 5 to 60 wt. %, particularly 20 to 60 wt. %, veneer materials or lateral and/or anterior tooth materials preferably 50 to 90 wt. % particularly 60 to 80 wt. %, filling composites preferably 50 to 90 wt. % particularly 75 to 85 wt. %, relative to the total weight of the dental material.

Furthermore, the dental materials in accordance with the invention can contain additional substances that are common in dental materials, e.g., from the group of pigments, stabilizers, antimicrobial additives, UV-absorbers, thixotropic agents, catalysts, photoinitiators and curing agents.

Such additives are used preferably in small quantities, in total 0.01 to 30, particularly 0.01 to 1.0 wt. %, relative to the total weight of the dental material.

The curing of the composites can occur by thermal, photochemical or redox-induced radical polymerization depending on the type of the polymerization initiator used.

Preferred examples of thermal initiators are the known peroxides such as, for example, benzoyl peroxide, dilauryl peroxide, tert.-butyl peroctoate or tert.-butylbenzoate and also azobisisobutyro ethyl ester, azobisisobutyronitrile, azobis-(2-methylpropionamidine)dihydrochloride, benzopinacol or 2,2-dimethylbenzopinacol.

Preferred photoinitiators are benzophenone, benzoin and their derivatives or alpha-diketones or their derivatives such as 9,10-phenanthrene quinone, diacetyl or 4,4-diclorobenzyl. Camphorquinone and 2,2-dimethoxy-2-phenylacetophenone are used with particular preference as also alpha-diketones in combination with amines as reduction agents such as, for example, 4-(N,N-dimethylamino)-benzoic acid ester, N,N-dimethylaminoethylmethacrylate, N,N-dimethyl-sym.-xylidine or triethanolamine. In addition, even acylphosphines such as, for example, 2,4,6-trimethylbenzoyldiphenyl- or bis(2,6-dichlorobenzyl)-4N-propylphenyl phosphine oxide are particularly suitable.

Preferred initiators for the polymerization that is performed at room temperature include redox-initiator combinations such as, e.g., combinations of benzoyl peroxide or lauryl peroxide with N,N-dimethyl-sym.-xylidine or N,N-dimethyl-p-toluidine.

The dental material in accordance with the invention can, e.g., be used in all types of filling composites, veneers, dental lacquers, fissure sealants, fastening cements, prostheses base materials, crown veneer composites and bridge veneer composites, adhesives or materials for artificial teeth. Accordingly the invention also concerns dental materials from the group of filling composites, fastening cements, prostheses base materials, crown veneer composites, dental lacquers, artificial teeth and adhesives that contain dispersions of nanoparticles that have an average particle size of 1 to <10 nm and are not agglomerated.

The invention is elucidated on the basis of the following example:

### EXAMPLE

In the first step, nanoscale SiO₂ particles with an average particle size of 4 nm are dispersed into a monomer mixture consisting of the components

| | |
|---|---|
| Bis-GMA | 17.5 wt. % |
| TEDMA | 7.5 wt. % |

The concentration of SiO₂ particles is selected such that in the end product there is a concentration of 10 wt. % SiO₂ after the addition of all other components.

The acrylate dispersion is subsequently added to 0.01 wt. % stabilizer BHT (4-methyl-2,6-di-tert-butyl-phenol, CAS 128-37-0) and also the initiators camphorquinone (0.3 wt. %) and 0.2 wt. % 2-ethylhexyl-4-dimethylaminobenzoate (Quantacure™ EHA, CAS 21245-02-3, Great Lakes Chemicals).

Addition of a dental glass (Ba—Al-silicate; 64.5 wt. %) and inner mixture yield a material that is suitable for use as a filling composite.

1. Dental material comprising an aqueous and/or organic solvent or dispersion, said solvent or dispersion comprising dispersed nanoparticles that have an average particle size of 1 to <10 nm and are not agglomerated.

2. Dental material in accordance with claim 1, wherein the nanoparticles comprise SiO₂ and/or ZrO₂ and/or Al₂O₃ and/or TiO₂.

3. Dental material in accordance with claim 1, wherein the solvent or dispersion is not aqueous.

4. Dental material in accordance with claim 1, which further comprises at least one filler selected from the group of dental glasses.

5. Dental material in accordance with claim 1, which further comprises agglomerated silicic acid.

6. Dental material in accordance with claim 1, wherein the organic solvent or dispersion is a mono- or polyfunctional acrylate or methacrylate.

7. Dental material in accordance with claim 1, which comprises oxide particles present in the form of their mixed oxides.

8. Dental material in accordance with claim 1, wherein the nanoparticles have surface modification.

9. Dental material in accordance with claim 1, wherein the nanoparticles are present in a monodisperse form.

10. Dental material in accordance with claim 1, which has a form selected from the group consisting of filling composites, fastening cements, prostheses base materials, dental lacquer, materials for artificial teeth, crown veneer composites, bridge veneer composites and adhesives.

11. Dental material in accordance with claim 1, which comprises ZrO₂ strengthened with Y₂O₃.

12. Dental material in accordance with claim 1, which comprises:
at least one monomer of the group Bis-GMA or TEDM (Triethylene glycol dimethacrylate)
a filler mixture from the groups
ZrO₂— and/or SiO₂ nanoparticles and dental glasses,
Al₂O₃ and/or SiO₂ nanoparticles and dental glasses,
TiO₂ and/or SiO₂ nanoparticles and dental glasses,
ZrO₂ and SiO₂ nanoparticles,
TiO₂ and SiO₂ nanoparticles,
Al₂O₃ and SiO₂ nanoparticles,
SiO₂ nanoparticles,
ZrO₂ nanoparticles,
Al₂O₃ nanoparticles,
TiO₂ nanoparticles.

13. Dental material in accordance with claim 1, wherein the nanoparticles have an average particle size of 1 to 8 nm.

14. Dental material in accordance with claim 1, wherein the nanoparticles have an average particle size of 2 to 6 nm.
